(19) United States
(12) Patent Application Publication
(10) Pub. No.: US 2008/0091073 A1
(43) Pub. Date: Apr. 17, 2008

(54) INFLATABLE ACTUATION DEVICE

(76) Inventor: Chul Hi Park, Los Altos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/872,025

(22) Filed: Oct. 14, 2007

Related U.S. Application Data
(60) Provisional application No. 60/852,316, filed on Oct. 16, 2006.

(51) Int. Cl. A61B 1/005

(52) U.S. Cl. 600/146

(57) ABSTRACT

An actuation device for an endoscope, the actuation device includes a tubular sheath configured to be provided around a shaft of the endoscope. An actuation bladder that is configured to be inflated and collapsed is provided between the tubular sheath and the shaft of the endoscope. The actuation device is configured to receive and manipulate the shaft of the endoscope.

### CROSS-REFERENCES TO RELATED APPLICATIONS

The present application claims the benefit of U.S. Provisional Application No. 60/852,316, filed on Oct. 16, 2006, which is incorporated by reference.

### BACKGROUND OF THE INVENTION

This invention relates to a device to assist in the insertion of a flexible endoscope into a tortuous passage and method of use. More particularly, it relates to an actuation device and method of use for manipulating the shaft of endoscope to facilitate insertion of a flexible endoscope into a tortuous bodily passage, such as the colon, and maneuver therein for the examination and treatment.

A flexible endoscope is a medical instrument of an elongate tubular shape for visualizing the interior of a patient's body. Flexible endoscopes can be used for a variety of different diagnostic and interventional procedures, including colonoscopy, sigmoidoscopy, bronchoscopy, thoracoscopy, laparoscopy and video endoscopy.

Colonoscopy is a medical procedure in which a flexible endoscope, or colonoscope, is inserted into patient's colon for diagnostic examination and/or surgical treatment of the colon. A standard colonoscope is typically 135-185 cm in length and 12-19 mm in diameter, and includes a fiber-optic imaging bundle or a miniature camera located at the instrument's tip, illumination fibers, one or two instrument channels that may also be used for insufflation or irrigation, air and water channels, and vacuum channels. A colonoscope is sufficiently stiff so that it does not buckle when it is pushed from outside the patient during insertion, yet flexible enough to be maneuvered through the tortuous lumen of the colon. The final six inches of the distal end of a colonoscope can be controlled by an endoscopist from outside the patient.

The most commonly used procedure for examining the colon is first to insert a colonoscope as far into the colon as desired while inspecting as the colonoscope advances. A detailed examination of the colon is made as the colonoscope is withdrawn. To examine the entire colon, the colonoscope is inserted through the anus into rectum, and then advanced through the sigmoid colon into the descending colon. The colonoscope then passes through the left colic flexure (the splenic flexure) into the transverse colon, and then through the right colic flexure (the hepatic flexure). The colonoscope next passes through the ascending colon and finally reaches the cecum.

To insert and advance a colonoscope into the colon, an endoscopist must employ a number of elaborate maneuvers to negotiate numerous bends and turns normally found in the colon. The only portion of a colonoscope shaft that can be maneuvered by a endoscopist is about 15 cm long portion at distal tip of the instrument, called the bending section, that can be tilted by up to 180 degrees in four directions, up and down, left and right. To advance a colonoscope, an endoscopist grasps the instrument at a point outside the body near the anus and pushes it inwards while maneuvering the bending section so that the tip of the instrument is pointed toward the direction of open lumen. Often the direction the tip of the instrument is pointing is not aligned with the direction of the shaft just behind the bending section. When endoscopist advances the instrument expecting the instrument to head in the direction the tip is pointed to, it frequently moves in an unanticipated direction instead and the tip ends up bumping into the colon wall. The end result of this maneuver is a “red-out,” a phenomenon named after the reddish hue filling up a monitor that displays images taken by a camera at the tip of the colonoscope. To recover from this misdirection endoscopist must pull back the instrument, steer the bending tip toward open lumen and try advancing the instrument again. Considerable time and efforts are spent in repeating this frustrating maneuver as endoscopist negotiates numerous bends in the colon in a typical colonoscopy procedure.

Repeated applications of steering and advancing maneuvers described above often leaves a crooked and/or a loop-like formation in the shaft of endoscope. This formation tends to be enlarged whenever the advancing portion of shaft or tip of endoscope is confronted with an obstacle such as a sharp bend or other form of resistance such as friction between the shaft and the colon wall. If not dealt with, the enlargement of these formations are bound to become a main cause of patient pain and serious difficulties in advancing the instrument into deeper part of the colon. To undo this undesirable formation and to straighten the shaft of endoscope, endoscopist pulls back while twisting the instrument in counter-clockwise or clockwise direction to remove a crooked or looped configuration in the shaft after the tip of endoscope enters the descending colon or passes the splenic flexure. The portion of the colon that has been threaded by the shaft of endoscope prior to this maneuver gets effectively pleated over the shortened and straightened shaft of endoscope that remains inside the colon. This maneuver generally requires fairly high level of skills on endoscopist's part and usually causes severe discomforts to the patient. Even for an experienced endoscopist this maneuver is a time consuming process and often must be repeated multiple time before a desired result can be obtained partially because endoscopist has no means to verify the progress and results of the maneuvers.

The formation of a loop in the shaft of a colonoscope is a common occurrence during intubation of the colon. In some people the sigmoid colon can be very long and is unfixed, except by its mesentery, and so can be extremely difficult to intubate due to its predisposition to form loops when a colonoscope is pushed through it. Some anatomical landmarks, such as rectosigmoidal junction, splenic flexure and hepatic flexure, are difficult to pass through simply because of their tortuous nature with present state of the art in endoscope technology. Problems traversing these areas are exacerbated by looping of the colonoscope in the sigmoid colon and subsequent stretching of the sigmoid colon causing discomfort for the patient undergoing the procedure.

Levy (U.S. Pat. App. No. 20060183974, incorporated herein in entirety with references therein) discloses an endoscope with an insertion tube fitted with an optical head. The insertion tube is coupled with a major inflatable sleeve and auxiliary inflatable sleeves, which upon inflation is capable of propelling the endoscope within the conduit. These inflatable sleeves are not capable of actively manipulating the insertion tube of the endoscope.

Chiel (U.S. Pat. App. No. 20030065250, incorporated herein in entirety with the references therein) discloses a self-propelled device capable of peristaltic locomotion. Peristaltic locomotion is caused by one or more actuators comprising an expandable bladder that surround a central flexible tube. A restorative spring placed inside the bladder restores the actuator to its original shape when not activated. Construction of this device is complicated and does not allow efficient bending of the instrument.

Gross (U.S. Pat. App. No. 20040102681, incorporated herein in entirety with the references therein) discloses a self-propelled imaging device. The imaging device comprises a carrier tube with first and second outwardly expandable elements. The forward movement of the device is driven by internal pressure in the second expandable element, which makes it expand and interact with the wall of the body lumen. This device propels the carrier tube but does not actuate the carrier tube itself.

There is a need to provide a device to solve these and other problems of colonoscopy.

### BRIEF SUMMARY OF THE INVENTION

The present invention relates to an inflatable device to assist in the insertion of a flexible endoscope into a tortuous passage and maneuver therein. An embodiment of the invention is directed to an actuation device and method of use for actuating and manipulating the shaft of a flexible endoscope to facilitate insertion into a tortuous bodily passage, such as the colon, and maneuver therein of a flexible endoscope for examination and treatment. The present actuation device generally comprises an actuation member and a tubular sheath. Alternatively, the present device may further comprise an inner, tubular sleeve of flexible material that receives the endoscope shaft and around which the actuation and tubular sheath are fixedly attached to aid in the deployment into the colon. Although the embodiments described in this specification specifically refers to the colon and colonoscopy procedure, the scope of their applicability is not limited to any particular bodily organ, other non-bodily tortuous passage or procedure.

In one embodiment, the actuation member generally comprises a collapsible, bladder of substantially annular configuration. Alternatively, the actuation member may comprise a plurality of collapsible bladders arranged side by side into a substantially annular configuration. Preferably, in a deflated state, the bladders are soft enough and of such a configuration to be pleated into a low profile form. Preferably, the bladders are substantially non-compliant. Alternately, the bladders may be substantially semi-compliant. These bladders are referred to as actuation bladders hereinafter.

In one embodiment, the tubular sheath generally comprises a collapsible, shaped, tube consisting of a body section and clamp sections. Preferably, the tubular sheath is substantially non-compliant. Alternately, the tubular sheath may be substantially semi-compliant. The body section may comprise a shaped, thin-walled tube. Alternatively, the body section may comprise a shaped, thin-walled tube with hole or pleat patterns of a predetermined configuration. Alternatively, the body section may comprise a mesh of a shaped, tubular configuration. The clamp sections are integral parts of the body section disposed at distal and proximal ends thereof. The clamp sections generally comprise tubular sleeves. The clamp sections further comprise inflatable, clamp bladders of substantially annular configurations fixedly disposed on the inner surfaces of the tubular sleeves. Preferably, the clamp bladders are substantially non-compliant. When fully inflated, the clamp bladders frictionally engage the shaft of endoscope. In one embodiment, the clamp sections may comprise welded joints between the tubular sheath and the inner tubular sleeve at distal and proximal ends of the body section. The tubular sheath is configured so that the body section substantially encloses the actuation bladders comprising the actuation member.

The inflatable actuation device of the present invention actuates or applies forces to a portion of the shaft of a flexible endoscope between two clamp sections of the tubular sheath to effect bending or straightening of the shaft. The source of energy needed for actuation is provided by the inflation pressure of the actuation member. The pressure inside the actuation member is built up by a pressurizing fluid supplied externally from a source outside the passage. The actuation bladders inflate freely until they meet the tubular sheath. The inflation stops when tensions on the tubular sheath created by inflating actuation bladders match the force applied by the internal pressure inside the actuation bladders. The tension forces are transmitted through the clamp sections of the tubular sheath to the shaft of endoscope which the clamp sections fixedly engage when fully inflated.

The inflatable actuation device of the present invention is capable of performing a variety of mechanical actuations and other functions working alone or in tandem. A bent section of the shaft of a flexible endoscope can be straightened by inflating all actuation bladders comprising the actuation member to equal internal pressure with the tubular sheath locked in position to span over a section of the bent shaft by fully inflating clamp bladders. The present actuation device is also capable of guiding a flexible endoscope while suppressing bending of the shaft of a flexible endoscope by maintaining the actuation member in a pressurized state and the clamp sections of the tubular sheath in a deflated state disengaged from moving shaft of endoscope. Alternatively, the present actuation device can bend a portion of the shaft of a flexible endoscope within the tubular sheath locked in position over a section of the endoscope shaft by fully inflating clamp bladders, by inflating only a selected actuation bladder or several actuation bladders, but not all, in coordination. Applied near the distal bending section of a flexible endoscope with the bending section locked in a straightened position, the present device can be used to perform wide-angle sweeps of the straightened bending section to unravel numerous bends in the colon, which are tricky to negotiate by flexing the bending section alone, and to actively seek out open lumen. This maneuver keeps the shaft of endoscope substantially straight and, at the same time, pleats the colon as the distal tip of endoscope is advanced.

By combining a plurality of the inflatable actuation devices of the present invention an actuation or guide device of a fairly complicated configuration and functionality may be constructed. The component actuation devices comprising the actuation or guide device may be arranged side by side or with predetermined gaps between neighboring devices.

For the purposes of this disclosure, including the appended claims, the terms “distal”, “distally”, and “distal end”, as they relate to the devices and methods described herein, refer to the end of the device further from or in the direction away from an operator who might be applying the device or method to the subject. Stated otherwise, the terms refer to the end of the device closer to or in the direction towards the patient's interior.

The terms “proximal”, “proximally”, and “proximal end”, as they relate to the devices and methods described herein, refer to the end of the device closer to or in the direction towards the operator who might be applying the device or method, rather than the patient.

### BRIEF DESCRIPTION OF THE DRAWINGS

The following exemplary figures are provided to supplement the description below and more clearly describe the invention. In the figures, like elements are generally designated with the same reference numeral for illustrative convenience and should not be used to limit the scope of the present invention.

FIG. 1A is a schematic, perspective view of an exemplary configuration of an inflatable actuation device mounted on the shaft of a flexible endoscope according to one embodiment of the present invention.

FIG. 1B is a schematic, perspective view of an exemplary configuration of an inflatable actuation device mounted on the shaft of a flexible endoscope according to another embodiment of the present invention.

FIGS. 2A and 2B are schematic, perspective views of exemplary configurations of an actuation member comprising a single bladder and a plurality of bladders, respectively, in an inflated state mounted on the shaft of a flexible endoscope according to one embodiment of the present invention.

FIG. 3 are a schematic, perspective view of an exemplary configuration of a tubular sheath with hole or pleat patterns according to one embodiment of the present invention.

FIGS. 4A and 4B are sectional views, along the symmetry plane of the shaft of an endoscope, of exemplary configurations of the inflatable actuation device with a bladder or all bladders comprising the actuation member, respectively, in an inflated state according to one embodiment of the present invention.

FIG. 5 is a sectional view, along the symmetry plane of the shaft of an endoscope, of an exemplary configuration of the inflatable actuation device with only one of the bladders comprising the actuation member inflated according to one embodiment of the present invention.

FIG. 6 is a schematic, perspective view of an exemplary configuration of a plurality of the inflatable actuation devices in various actuation states according to one embodiment of the present invention.

FIG. 7 is a schematic, perspective view of an exemplary configuration of a mesh sleeve of a tubular configuration according to one embodiment of the present invention.

### DETAILED DESCRIPTION OF THE INVENTION

Embodiments of the present invention relate to an inflatable actuation device capable of manipulating the shaft of a flexible endoscope to assist in the insertion of an endoscope into a tortuous passage and a method of use thereof.

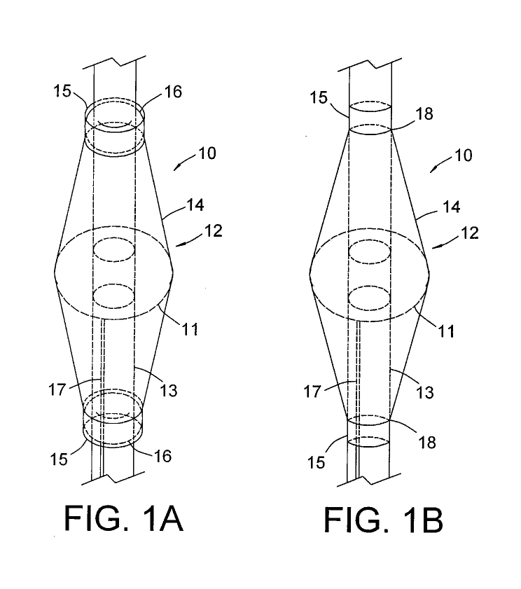

Referring to FIG. 1A, the inflatable actuation device 10 comprising an actuation member 11 in an inflated state and a shaped, tubular sheath 12 including a body section 14 and clamp sections 15, disposed around the shaft 13 of an endoscope according to one embodiment of the present invention is shown. Also shown are clamp bladders 16, further comprising clamp sections, fixedly disposed on the inner surface of a tubular sleeve comprising clamp sections 15 and a supply tube 17 for supplying pressurizing fluid to actuation member 11. Supply tubes for clamp bladders 16 are omitted in the figure for clarity. These components comprising inflatable actuation device 10 may be fixedly mounted on a flexible inner sleeve (not shown) of substantially tubular shape, which receives the endoscope shaft, to facilitate deployment in and retrieval from the colon. In one embodiment, clamp bladders 16 may be substituted with fixed joints 18 between the clamp sections and the flexible inner sleeve as schematically shown in FIG. 1B. Any suitable joining method may be used, for example, mechanical joint, thermal bonding or adhesive bonding. In one implementation, the flexible inner sleeve may be made of a thermoplastic material. Alternately, the flexible sleeve may be made of an elastomeric material of a predetermined elasticity. The flexible sleeve may be reinforced with a braid of a predetermined material and configuration to enhance its compressive strength in axial direction and kink resistance.

During initial insertion into and withdrawal of the actuation device 10 from the colon, actuation member 11 and clamp bladders 16 may be in a deflated and pleated state in a low profile form. Tubular sheath 12 is also pleated into a low profile form to facilitate insertion through the anus. Actuation device 10 may be held to the shaft of endoscope 13 during insertion into the colon by proximal and distal fixation mechanisms (not shown). The fixation mechanisms may comprise bands of resilient, flexible material fixedly disposed encapsulating proximal and distal ends of the actuation device 10 and the shaft of endoscope. Alternatively, the bands may be releasably disposed.

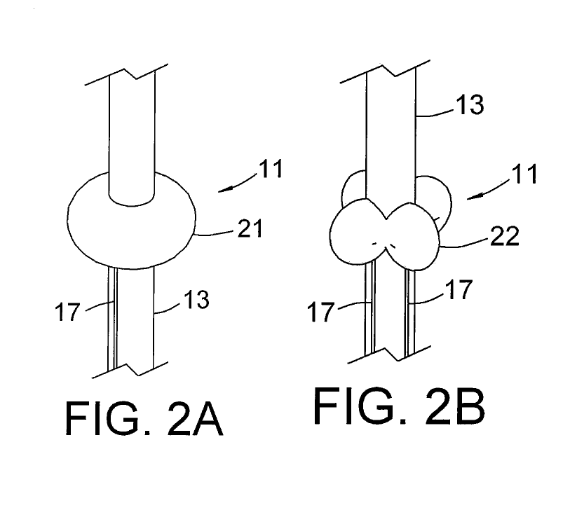

FIG. 2A is a schematic perspective view of an actuation member 11 comprising a substantially annular actuation bladder 21 according to one embodiment of the present invention. Preferably, the inner diameter of annular actuation bladder 21 is substantially equal to or slightly larger than the diameter of shaft 13 of endoscope, for example, between 5 mm and 15 mm, so that the endoscope can travel freely through the center hole of actuation bladder 21. The outer diameter of annular actuation bladder 21 in an inflated state, preferably, is predetermined to be large enough to come in contact with and apply forces to body section 14 of tubular sheath 12, as shown in FIG. 1A but smaller than general transverse dimension of a passage where the inflatable actuation device of the present embodiment is to be deployed. Preferably, the outer diameter may be, for example, between 1 cm and 6 cm. The length of actuation bladder 21 along the axis of shaft 13 may be predetermined, for example, between 1 cm and 5 cm, in consideration of the length of tubular sheath 12 as shown in FIG. 1A and the mechanical characteristics of shaft 13. In addition to being a source of actuation force, actuation bladder 21 in an inflated state may also function as a brace supporting and keeping shaft 13 in a substantially straight configuration when subjected to transverse loads at positions along the shaft outside the length covered by inflatable actuation device 10. In an embodiment represented in FIG. 2A the inflatable actuation device of the present embodiment is capable of straightening a curved portion of the shaft of a flexible endoscope enclosed therein and maintaining it in straight position.

FIG. 2B illustrates an actuation member 11 comprising a plurality of actuation bladders 22 according to one embodiment of the present invention. Actuation ladders 22 are of substantially equal in size and mounted, fixed in position relative to one another, on a flexible sleeve fitted around the endoscope shaft arranged substantially evenly in a circular pattern to form an annular-like configuration around the sleeve. Alternatively each actuation bladder 22 may have different sizes from each other and/or be arranged unevenly. Overall sizes of actuation bladders 22 may be comparable to that shown in FIG. 2A. The number of actuation bladders 22 comprising actuation member 11 may be between 2 and 8, preferably, 3 and 6. Internal pressure of actuation bladders 22 may be controlled independently from one another. Alternately, all actuation bladders may be configured to be in fluid communication with one another and be inflated through a single supply tube. With their internal pressures controlled in unison, actuation bladders 22 function in much the same way as a single annular bladder as described in accordance with the embodiment shown in FIG. 2A. Alternately, by controlling their internal pressures independently from one another, actuation bladders 22 can be used to effect a bending of shaft 13 in a predetermined direction as will be described in more detail in conjunction with FIG. 5 below.

Still referring to FIGS. 2A and 2B, there are provided openings near the proximal ends of actuation bladders 21, 22 comprising actuation member 11. The openings are sealingly attached to and in fluid communication with distal ends of supply tubes 17. The proximal ends of the supply tubes are fixedly and sealingly attached to supply ports of a supply tube assembly (not shown). The supply tube assembly is held in fixed position near the anus. There are provided conduit in the supply tube assembly through which a fluid may pass to and from between a pressure controller unit external to the colon and the actuation bladders to control the internal pressure thereof. Alternately, the supply tube may be attached directly to an output port of a pressure controller unit and is free to move in and out of the colon. Preferably, the fluid is a low viscosity liquid (e.g., water or saline solution). Alternatively, it may be a gas (e.g., air, nitrogen, or carbon dioxide).

The actuation bladders may be made of thin yet high tensile modulus polymer film material. Suitable materials may be, for example, polyethylene terephthalate (PET), polypropylene, polyamide (Nylon), polyimide (Kapton), polyvinylchloride (PVC), polyurethane and polyethylene. Alternately, the actuation bladders may be made of an elastomeric material of a predetermined elasticity. Any suitable method may be employed to construct the bladder member, for example, blow molding of a preconfigured thermoplastic polymer tube. In the present implementation, the wall thickness of the actuation bladders may be between 0.005 mm and 0.5 mm. The internal pressure may be between 0.2 atmosphere and 15 atmosphere above ambient pressure. In one implementation, the internal pressure is between 0.3 atmosphere and 3 atmosphere above ambient pressure. In another implementation, the internal pressure is more than 8 atmosphere above ambient pressure.

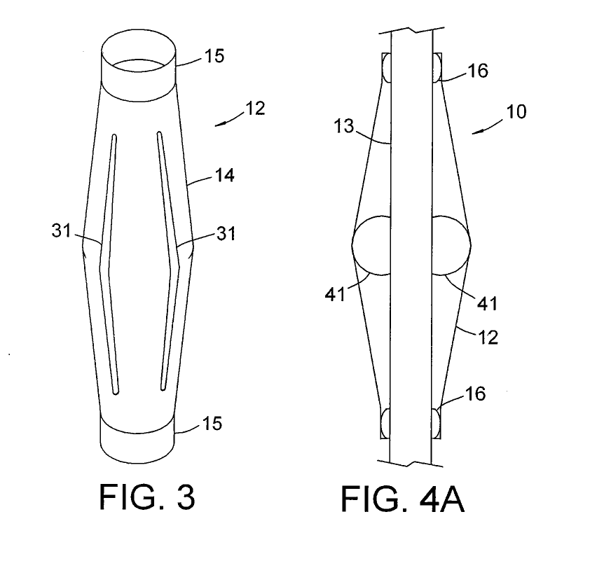

An embodiment of a shaped tubular sheath 12 is shown in FIG. 3. Tubular sheath 12 comprises body section 14 and clamp sections 15. Body section 14 may include holes or pleat patterns 31 to help it conform to the configuration of actuation bladders 21, 22 shown in FIGS. 2A and 2B and to facilitate folding it into a low profile form. Alternately, the tubular sheath may comprise a mesh of shaped, tubular configuration as shown in FIG. 7 in an exemplary configuration. Alternately, the tubular sheath may comprise a shaped tube reinforced with a mesh of a predetermined configuration.

In one implementation, for example, body section 14 comprising tubular sheath 12 may be divided into a plurality of strips of substantially equal width running parallel to the axis of body section 14. The number of strips may be equal to the number of actuation bladders comprising the actuation member. Each strip is disposed substantially centered with respect to an actuation bladder, in case the actuation member comprises a plurality of actuation bladders. Each strip may be configured to substantially fit the contour of contact surface of corresponding actuation bladder in an inflated state.

Tubular sheath 12 may be made of thin yet high tensile modulus polymer film material. Suitable construction materials may be, for example, polyethylene terephthalate (PET), polypropylene, polyamide (Nylon), polyimide (Kapton), polyvinylchloride (PVC), polyurethane and polyethylene. Alternately, the tubular sheath may be made of an elastomeric material of a predetermined elasticity. Any suitable method may be employed to construct the tubular sheath, for example, blow molding of a preconfigured thermoplastic polymer tube or an extrusion. A secondary process may be employed to provide hole or pleat patterns when needed. In the present implementation, the wall thickness of tubular sheath 12 may be between 0.005 mm and 0.5 mm. The mesh comprising the tubular sheath may be made of high tensile strength filaments of material, for example, polyethylene terephthalate (PET), polypropylene, polyamide (Nylon), polyimide (Kapton), polyvinylchloride (PVC), polyurethane and polyethylene. In one embodiment, tubular sheath or mesh comprising the tubular sheath may be of elastomeric material of a predetermined elasticity. Overall length of the tubular sheath and accordingly, the actuation device of the present embodiment may be determined to suit a given application, for example, between 2 cm and 20 cm, more particularly, 5 cm and 12 cm.

Still referring to FIG. 3, the largest circumference near the middle of body section 14 of tubular sheath 12 may not be larger than the outer diameter of annular configuration of actuation bladders comprising the actuation member in an inflated state. This is to allow the actuation bladders to come in contact with and apply tension to the tubular sheath as they are inflated before they reach a maximum design profile. Similarly, the outer diameter of tubular sleeve 15 comprising a clamp section is designed not to be larger than a maximum design diameter of the clamp bladder 16, as shown in FIG. 1A, comprising a clamp section in an inflated state, so that the clamp bladder can apply clamping force to the shaft of a flexible endoscope when pressurized. The lengths of the tubular sleeve and the clamp bladder may be predetermined to provide large enough clamp force to suit a given application in conjunction with internal pressure in the clamp bladder.

Openings are provided near the proximal ends of the clamp bladders comprising the clamp sections. The openings are sealingly attached to and in fluid communication with distal ends of supply tubes. The proximal ends of the supply tubes are fixedly and sealingly attached to supply ports of a supply tube assembly. The supply tube assembly is held in fixed position near the anus. A conduit is provided in the supply tube assembly through which a fluid may pass to and from between a pressure controller unit external to the colon and the bladders to control the internal pressure thereof. Alternatively, the supply tube may be attached directly to an output port of a pressure controller unit and is free to move in and out of the colon. Preferably, the fluid is a low viscosity liquid (e.g., water or saline solution). Alternatively, it may be a gas (e.g., air, nitrogen, or carbon dioxide).

Materials and production methods, and operating parameters comparable to those used for the actuation bladders may be used for the clamp bladders.

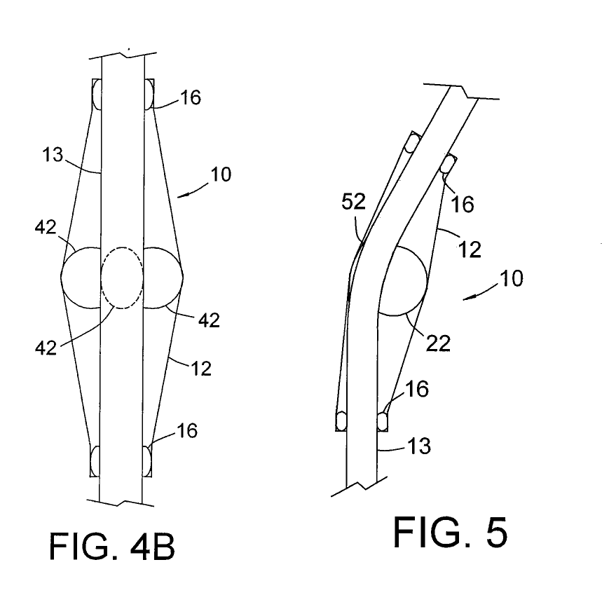

Referring to FIG. 4A is a sectional view, along the symmetry plane of the shaft 13 of an endoscope in a straightened position. Actuation bladder 41 is in an inflated state pulling on the tubular sheath 12 with equal force all around with respect to the axis of shaft 13. Any transverse load acting on the portion of shaft 13 outside tubular sheath 12 would be counteracted by the tension present on the sheath and not able to cause a bending to the portion of shaft 13 within tubular sheath 12. If a portion of the shaft of endoscope was in bent configuration, it will be straightened as the actuation bladders are inflated. Tubular sheath 12 is friction-locked to and held in position on shaft 13 of endoscope by clamp bladders 16 in an inflated state. Similar function can be performed by the actuation member 11 comprising a plurality of bladders 42 of substantially equal sizes, as shown in FIG. 4B, by inflating all actuation bladders 42 to an equal internal pressure. Supply tubes for the actuation and clamp bladders are omitted in the figures for clarity.

FIG. 5 schematically illustrates an exemplary method of actuation of the inflatable actuation device 10 comprising a plurality of actuation bladders, where some actuation bladders are inflated 22 and others are left in deflated state 52 to cause a bending of endoscope shaft. The viewpoint of the illustration is similar to that of FIGS. 4A and 4B, which is a sectional view, along the symmetry plane of the shaft 13 of a flexible endoscope. The clamp bladders 16 are fully inflated to grip the shaft 13 of endoscope and keep the tubular sheath 12 from sliding down shaft 13 when actuation bladder 22 is inflated and pulls on tubular sheath 12. The bladder 52 disposed substantially opposite to inflated actuation bladder 22 with respect to shaft 13 may be left in a deflated state or inflated to a predetermined pressure lower than that of actuation bladders 22 to control the degree of bending. Other bladders that are not directly opposite to the inflated actuation bladders may be inflated to a given pressure to render a side support to shaft 13 to suppress it from bending in a direction perpendicular to the intended bending direction or they may be left in a deflated state. Shaft 13 bends in the direction of inflated actuation bladder 22 due to disproportionately higher tension on a portion of tubular sheath 12 in contact with actuation bladder 22. The degree of bending may be controlled by adjusting pressures difference between actuation bladders 22, 52. Supply tubes are omitted in the figures for clarity.

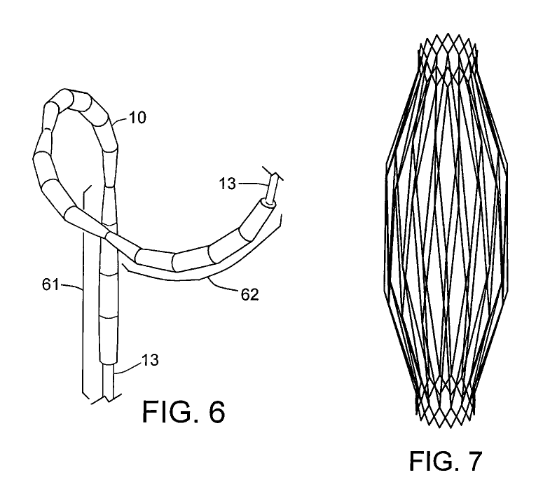

FIG. 6 illustrates a schematic, perspective view of an exemplary configuration of a plurality of the inflatable actuation devices 10 disposed around the shaft 13 of an endoscope in various actuation states according to one embodiment of the present invention. By appropriately activating inflatable actuation devices 10 in a predetermined sequence, a straight 61 or a curved 62 configuration or a complex configuration combining straight and curved sections in arbitrary sequence may be rendered to shaft 13 of a flexible endoscope.

The inflatable actuation device described above may be implemented various ways to insert and maneuver a flexible endoscope in a tortuous bodily passage. In one implementation, the inflatable actuation device may be used to effect a sweeping motion of the distal portion near the bending section of the shaft of a flexible endoscope to assist in the navigation of the tip of endoscope through the tortuousness of the colon, particularly, the sigmoid colon. With the inflatable actuation device fixedly disposed adjacent to the proximal end of the bending section and the bending section locked in straightened position, repeated bending actuation in opposing directions by the present device effectively results in sweeping motion of the straightened bending section.

In another implementation the inflatable actuation device may be deployed at predetermined positions in the colon, released from the endoscope and tethered to the supply tubes to remain stationary with respect to moving shaft of endoscope, after being introduced into the colon mounted on the shaft of endoscope to which it is releasably attached. After activated and fixed to a predetermined configuration the actuation device may be used to guide the shaft of endoscope. For example, the present actuation device may be deployed around a bend in the colon, e.g., the splenic flexure, to make the shaft to conform to the bend around which it is being advanced.

While preferred illustrative embodiments of the invention are described above, it will be apparent to those skilled in the art that various changes and modifications may be made therein without departing from the invention. Accordingly, the appended claims should be used to interpret the scope of the present invention.

What is claimed is:

1. An actuation device for an endoscope, the actuation device comprising:
a tubular sheath configured to be provided around a shaft of the endoscope; and
an actuation bladder that is configured to be inflated and collapsed and provided between the tubular sheath and the shaft of the endoscope, wherein the actuation device is configured to receive and manipulate the shaft of the endoscope.

2. The device of claim 1, further comprising:
a first clamp bladder provided on one end of the tubular sheath and a second clamp bladder provided on the other end of the tubular sheath.

3. The device of claim 1, further comprising:
a first fixed joint provided on one end of the tubular sheath and a second fixed joint provided on the other end of the tubular sheath.

4. The device of claim 1, further comprising:
a flexible sleeve provided between the actuation bladder and the shaft of endoscope.

5. The device of claim 1, wherein a plurality of actuation bladders are provided between the tubular sheath and the shaft of the endoscope.

6. The device of claim 5, wherein the plurality of the actuation bladders are all of substantially equal size.

7. The device of claim 6, wherein the plurality of the actuation bladders are provided in fixed positions relative to each other.

8. The device of claim 5, wherein the plurality of the actuation bladders are of different sizes.

9. The device of claim 5, wherein the plurality of the actuation bladders are substantially non-compliant.

10. The device of claim 5, wherein the plurality of the actuation bladders are substantially semi-compliant.

11. The device of claim 5, wherein internal pressures of the plurality of the actuation bladders are controlled to bend the shaft in a given direction.

12. The device of claim 1, wherein the tubular sheath includes one or more pleated patterns on a body section of the tubular sheath.

13. The device of claim 1, wherein the tubular sheath has a mesh-shaped, tubular configuration.

14. The device of claim 1, wherein the tubular sheath comprises a shaped tube reinforced with a mesh of a predetermined material and configuration.

15. The device of claim 1, wherein the length of the actuation device is between 2 cm and 20 cm.

16. The device of claim 1, wherein the length of the actuation device is between 5 cm and 12 cm.

17. An actuation device for an endoscope, the actuation device comprising:
a tubular sheath configured to be provided around a shaft of the endoscope; and
an actuation member provided between the tubular sheath and the shaft of the endoscope, the actuation member being configured to control the bending of the shaft,
wherein the actuation device is configured to receive and manipulate the shaft of the endoscope.

18. The device of claim 17, wherein the actuation member includes at least one actuation bladder that is configured to be inflated or collapsed.

19. A method of navigating an endoscope within an organ, the method comprising:
providing the endoscope with a tubular sheath disposed around a shaft of the endoscope and an actuation member provided between the tubular sheath and the shaft;
inserting the shaft of the endoscope and the tubular sheath into the organ; and
bending the shaft of the endoscope by using the actuation member provided between the tubular sheath and the shaft of the endoscope.

20. The method of claim 19, wherein the actuation member includes at least one actuation bladder, wherein the shaft of the endoscope and the tubular sheath are inserted into the organ while the actuation bladder is kept a deflated state.

21. The method of claim 20, wherein bending the shaft of the endoscope is achieved by inflating the actuation bladder to a given pressure.
